(19) United States
(12) Patent Application Publication
(10) Pub. No.: US 2008/0177357 A1
(43) Pub. Date: Jul. 24, 2008

(54) UV LIGHT IRRADIATION MACHINE FOR VETERINARY USE

(76) Inventor: Thomas Perez, Chicago, IL (US)

Correspondence Address: ADAM K. SACHAROFF;MUCH SHELIST FREED DENENBERG AMENT&RUBENSTEIN,PC, 191 N. WACKER DRIVE, SUITE 1800, CHICAGO, IL, 60606-1615, US

(21) Appl. No.: 12/033,517

(22) Filed: Feb. 19, 2008

Related U.S. Application Data
(63) Continuation-in-part of application No. 11/125,789, filed on May 10, 2005, now abandoned.

(51) Int. Cl. A61B 18/18

(52) U.S. Cl. 607/91; 606/2

(57) ABSTRACT

Ultraviolet light is applied to an animal's blood while that blood remains in the body. The UV light is applied sublingually or directly to the blood. One of more light guides is used in connection with a bridle to help directed the ends of the light guide under the animal's tongue.

### CROSS REFERENCE TO RELATED APPLICATIONS

The present application is a Continuation-In-Part Application of U.S. patent application Ser. No. 11/125,789.

### FIELD OF THE INVENTION

The present invention relates to a method and apparatus for sublingual application of UV light to animals.

### BACKGROUND OF THE INVENTION

Ultraviolet (UV) light can be used to treat a multitude of medical problems in humans and animals, including for example bacterial, viral and fungal infections, poisoning, fatigue, allergies and asthma, rheumatic diseases and arthritis, diabetes, hepatitis, and cancer. UV light sterilizes the blood and acts as an antibiotic.

The UV light is applied either to the patient's skin or directly to the blood. If the UV light is applied to the skin it is typically provided to the patient's skin either with a wrap or lamp. For example, UV light is commonly used to treat jaundiced babies, because infant's skin is thin and the blood vessels are close to the surface. A wrap of lamp is generally unsuitable for treating animals because animal skin is often thick and/or coated with fur and/or there is a fat layer under the skin. Thus, the capillaries are often not close to the surface.

The other method as mentioned above is to apply the UV light directly to the blood. Applying the UV light directly to a patient's blood supply is known as photoluminescence or UV blood illumination (UBI). UBI increases oxygen, destroys toxins, and boosts the immune system. In prior art UBI for humans, a small amount of blood is drawn from the patient, up to about 250 cc. The blood that is drawn travels through a cuvette or glass chamber. The blood is repeatedly illuminated with UV light and then returned to the body. The process is repeated, typically a day or several days later. These treatments are time consuming and require regular trips to a medical facility. As one can imagine applying this method to an animal would be extremely difficult.

There is thus a need for a method of providing UV light to animals. There is a need for system which would allow the animal's blood to be illuminated without removal from the body. There is a need for a system that allows the blood to be treated on an as needed basis, such as based on the animals current symptoms at a particular time or response to the treatment.

### SUMMARY OF THE INVENTION

The present invention includes in one embodiment a system for delivering UV light to an animal. The system includes a device having a UV light source and a bridle capable of being secured around a head of the animal. The device includes at least one light guide having a first end secured to the device for receiving the UV light and a second end adapted to emit the UV light. The bridle has a noseband adapted for placement around a mouth of the animal. At least one opening in the noseband is cooperative with the light guide. The opening has a radius sized to receive the second end of the light guide and may be positioned on the noseband such that when the bridle is secured around the head of the animal the second end is posed for placement under a tongue of the animal. The bridle when secured around the head of the animal and when the light guide is placed through the opening in the noseband and under the tongue of the animal, places the second end of the light guide in a position to deliver the UV light to the animal. The UV light would be delivered to the animal under the tongue which allows the UV light to be absorbed into the animal's blood.

The bridle may further include a crown piece adapted for placement around the head of the animal. The crown piece is attached to the noseband by a cheek strap. The cheek strap may include a headstall adapted for placement around a back portion of the head and may include a latch cooperative with ends defined by the cheek strap for adjusting the length of cheek strap. Lastly, the bridle may further include a connection member positioned in each of said opening for holding the light guides within said opening.

In another embodiment, a method for delivering UV light to an animal is defined. The method includes providing a source of UV light, delivering the UV light through a light guide to an end defined to emit the UV light, securing a bridle around a head of the animal, and positioning the end of the light guide through an opening defined in a noseband such that the end of the light guide is positioned and placed under a tongue of the animal such that UV light is emitted to the animal under the tongue.

Numerous advantages and features of the invention will become readily apparent from the following detailed description of the invention and the embodiments thereof, and from the accompanying drawings.

### BRIEF DESCRIPTION OF THE DRAWINGS

A fuller understanding of the foregoing may be had by reference to the accompanying drawings, wherein:

FIG. 1 is a perspective view of an UV light irradiation delivery device used in connection with an animal bridle in accordance with an embodiment of the invention;

FIG. 2 is a perspective view showing the tongue of an animal with light guides placed there under for a sub-lingual UV light irradiation delivery system;

FIG. 3 is an animal bridle in accordance with an embodiment of the invention;

FIG. 4 is another animal bridle in accordance with another embodiment of the invention; and

FIG. 5 is yet another animal bridle in accordance with another embodiment of the invention.

### DETAILED DESCRIPTION OF THE INVENTION

While the invention is susceptible to embodiments in many different forms, there are shown in the drawings and will be described herein, in detail, the preferred embodiments of the present invention. It should be understood, however, that the present disclosure is to be considered an exemplification of the principles of the invention and is not intended to limit the spirit or scope of the invention and/or the embodiments illustrated.

Ultraviolet light (UV) can is used to treat many diseases including bacterial, viral, and fungal infections, poisoning, fatigue, allergies, hepatitis, cancer and HIV. UV light increases the oxygen combining power of the blood, destroys toxins, viruses, fungi, bacteria, and boosts the immune system. UV light also sterilizes the blood and acts as an antibiotic. Preferably, the UV light is selected based on the condition being treated. For treatments UV-C is preferred, at wavelengths between 90-280 nm, and more preferably between 200-280 nm, and most preferably at a peak of 245 nm. While UV-C is preferred it may be possible to use UV-A, or UV-B or a combination of various UV wavelengths. Animals get many of the same diseases as humans as well as other diseases which need treatment. For example, horses or newborn colts may develop a bacterial infection for which the current treatment is very high doses of antibiotic. The horse is likely to die from the treatments. Cats get feline leukemia, feline HIV, and feline diabetes.

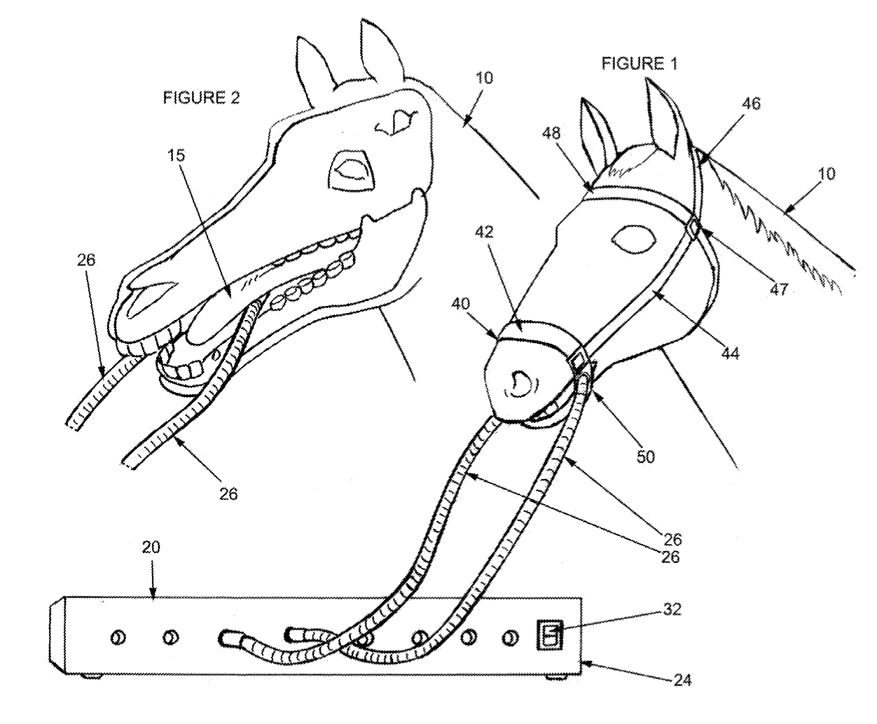

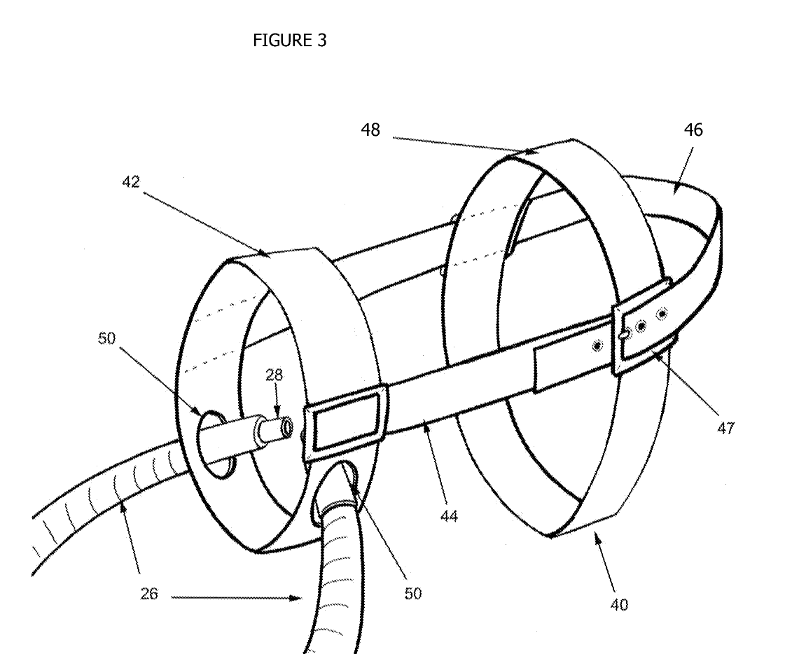

One embodiment of the present invention is shown in FIG. 1, which illustrates a UV device 20 that is connectable to an animal 10 by being inserted under the tongue 15 of the animal 20 (shown in FIG. 2. Since the capillaries under the tongue 15 are close to the surface, these capillaries are very sensitive. Capillary exposure of the mucus membrane is significantly greater than other exposed body surfaces. The greater capillary exposure allows for greater penetration of the ultraviolet spectrum into the bloodstream.

The UV device 20 comprises a housing 24, battery, power cable connectable to an outlet or generator, or other power source (not shown) and a UV light source. The UV light source may be in the housing or it may be placed at the point of illumination. Preferably, UV light source is LEDs emitting UV light at a therapeutic wavelength. Alternatively, a plurality of LEDs, having one or more different wavelengths of UV light could be used. The UV device 20 allows UV light to be supplied directly to the blood or to irradiate the blood supply very close to the surface. Thus, large amounts of blood or even the entire blood supply of an animal can be treated.

In one embodiment, the UV device 20 has at least one light guide 26 having a liquid core for transporting the UV light from the housing to the tip 28. The liquid core light guide may be of the type manufactured by Newport Corporation type DUV or UV-VIS liquid light guides. The outer material used on the light guide 26 may be selected based on the species of animal being treated, their propensity to damage or play with the light guide, the conditions under which the animal is being treated or based on other considerations. Alternatively to the liquid core light guide, fused silica fiber optic bundles may be use because they do not emit any heat. The fiber optic bundles would preferably deliver UV light sublingually. In yet another alternative, UV light source is a LED or small light bulb at the end of the flexible tube adapted to directly irradiate the mucus membrane under the tongue.

For treatments, one end 30 of the light guide is connectable to the housing and the other end is placed when the irradiation is desired. Emitted UV radiation travels to the end tip 28 of the light guide 22 and is emitted. Emitted radiation directly illuminates the blood or the area under the tongue. Sublingual administration of the UV radiation is preferred because the capillaries are close to the surface on the mucus membrane under the tongue. The UV light is thus able to treat the blood more effectively.

It is preferable if the light guide 22 is inserted into the animal's mouth 42 in an area where it will cause the least discomfort to the animal and where the animal is least likely to damage the light guide. For example, for a horse, the light guide 22 may be inserted on the side of the mouth rather than in the front. The number of light guides 22, intensity, therapeutic wavelength, and/or treatment times of the light is determined by the size of the animal 10, species, age of the animal, condition being treated and/or based on other variables.

The animal can be connected to the UV device 20 when a treatment is needed. The UV device 20 could be turned on and off manually with a switch 32 or by another know method. Alternatively, the UV device 20 could automatically turn off the light source after a set treatment time, such as after fifteen to twenty minutes. The UV device 20 could have a controller such as a computer or other interface that limits the number of treatments given time period, limits the total amount of treatment time in a given time period, automatically provides treatments, pulses the light source, or provides only particular wavelengths. The computer or other interface could keep a treatment record. The computer or other interface could communicate wirelessly, via the Internet or through other electronic means to automatically update the treatment records for that animal. The computer preferably can automatically adjust treatment time, wavelength, or other variables based on symptom.

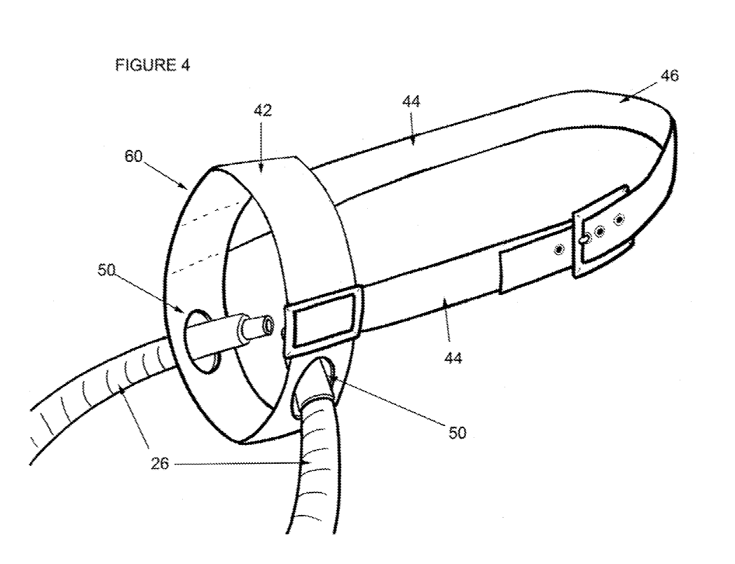

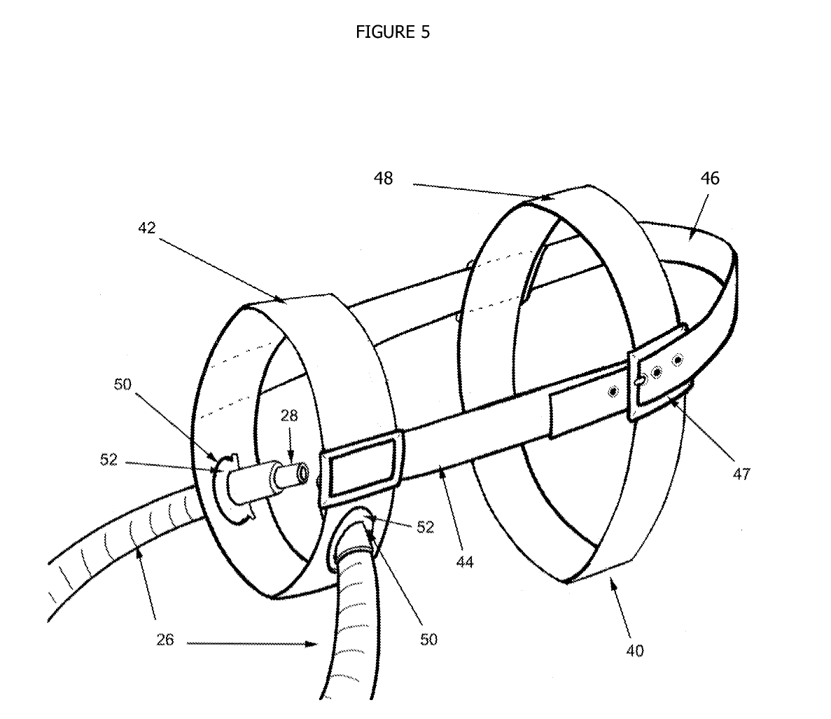

Referring now to FIGS. 1 and 3, to facilitate connection of the UV device under the tongue of the animal, the end tips 28 of the light guides 26 can be secured to a bridle arrangement 40. The bridle arrangement 40 is shown in FIG. 1 mounted to the head of a horse or similar animal 10. The bridle arrangement 10 is utilized to control not only the placement of end tips 28 of the light guides 26 but to help hold them in place during treatment. The bridle arrangement 40 generally comprises a noseband 42 with a pair of cheek straps 44 that secure to a headstall 46. The cheek straps 44 are preferably adjustable and include at least a latch 47. The bridle arrangement 40 may also include a crown piece 48 that would fit over the head of the animal. However, the crown piece 48 if not included (as shown in FIG. 4) would create a bridle arrangement 60 that is similar to a muzzle.

The noseband 42 includes a pair of opening 50 that have a radius sized to receive at least the circumference of end tips 28 of the light guides 26 and may also be sized to receive the circumference of the light guides 26 themselves. In addition, as shown in FIG. 5, the openings may further include a connection member 52 to help aid in holding the light guides 26 in place. The connection member 52 may include, but is not limited to a gasket, rubber O-ring, or webbing.

In another embodiment, the light guide 26 is inserted through the animal's nostril and irradiates the mucus membrane of the throat, stomach or other membrane with capillaries close to the surface. In another embodiment, UV device 20 is surgically attached to animal 10 or implanted in the animal 10. In such embodiments, the light guide 22 would include a flexible catheter and transparent cover at the end tip. In an alternative embodiment, light source such as a LED, or miniature light bulb located at the end tip of a guide tubing housing a power cord is connected to a power source.

From the foregoing and as mentioned above, it will be observed that numerous variations and modifications may be effected without departing from the spirit and scope of the novel concept of the invention. It is to be understood that no limitation with respect to the specific methods and apparatus illustrated herein is intended or should be inferred.

I claim:

1. A system for delivering UV light to an animal, the system comprising:
a device having a UV light source and at least one light guide, said light guide having a first end secured to the device for receiving the UV light and a second end adapted to emit the UV light; and
a bridle capable of being secured around a head of the animal, the bridle having a noseband adapted for placement around a mouth of the animal, the noseband having at least one opening cooperative with said light guide, said opening having a radius sized to receive said second end of said light guide, and said opening being positioned on the noseband such that when the bridle is secured around the head of the animal said opening is posed for placement of said second end of said light guide, being inserted therethrough, under a tongue of the animal.

2. The system of claim 1, wherein the bridle being secured around the head of the animal and placing said light guide through the said opening in the noseband of the bridle and under the tongue of the animal, places said second end of the light guide in a position to deliver the UV light to the animal.

3. The system of claim 2, wherein the UV light being delivered to the animal under the tongue is absorbed into the animal's blood.

4. The system of claim 1, wherein the bridle further includes a crown piece adapted for placement around the head of the animal, said crown piece is attached to the noseband by a cheek strap.

5. The system of claim 4, wherein the cheek strap of the bridle includes a headstall adapted for placement around a back portion of the head.

6. The system of claim 4, wherein the bridle further includes a latch cooperative with ends defined by the cheek strap for adjusting the length of cheek strap.

7. The system of claim 1, wherein the bridle further includes a connection member positioned in each of said opening for holding the light guides within said opening.

8. The system of claim 1, wherein the UV light source produced is UV-C light.

9. The system of claim 1, wherein the light guide has a liquid core for transporting the UV light from the first end to the second end.

10. A method for delivering UV light to an animal comprising:
providing a source of UV light;
delivering the UV light through a light guide to an end defined to emit the UV light;
securing a bridle around a head of the animal, the bridle being defined as having a noseband adapted for placement around a mouth of the animal, the noseband having at least one opening cooperative with said light guide, said opening having a radius sized to receive said end of said light guide; and
positioning said end of said light guide through said opening of said noseband under a tongue of the animal such that UV light is emitted to the animal under the tongue.

11. The method of claim 10, wherein the opening of said noseband is further positioned on the noseband such that when the bridle is secured around the head of the animal said second end is posed for placement under a tongue of the animal.

12. The method of claim 10 further comprising placing a crown piece defined by the bridle and attached to the noseband by a cheek strap around the head of the animal.

13. The method of claim 12 further comprising placing a headstall defined by the bridle as attached to the cheek strap of the bridle around a back portion of the head of the animal.

14. The method of claim 13 further comprising adjusting the length of the cheek strap by adjusting a latch defined between ends of the cheek strap.

15. A system for delivering UV light to an animal, the system comprising:
a device having a UV light source and at least one light guide, said light guide having a first end secured to the device for receiving the UV light and a second end adapted to emit the UV light; and
a bridle capable of being secured around a head of the animal, the bridle having a noseband adapted for placement around a mouth of the animal, the noseband having at least one opening cooperative with said light guide, said opening having a radius sized to receive said second end of said light guide, such that when the bridle is secured around the head of the animal said second end is posed for placement under a tongue of the animal.

16. The system of claim 15, wherein the UV light being delivered to the animal under the tongue is absorbed into the animal's blood.

17. The system of claim 15, wherein the bridle further includes a crown piece adapted for placement around the head of the animal, said crown piece is attached to the noseband by a cheek strap and wherein the cheek strap of the bridle includes a headstall adapted for placement around a back portion of the head.

18. The system of claim 17, wherein the bridle further includes a latch cooperative with ends defined by the cheek strap for adjusting the length of cheek strap.

19. The system of claim 15, wherein the bridle further includes a connection member positioned in each of said opening for holding the light guides within said opening.

20. The system of claim 15, wherein the UV light source produced is UV-C light.
